(19) United States
(12) Patent Application Publication
(10) Pub. No.: US 2026/0333521 A1
(43) Pub. Date: Oct. 22, 2026

(54) WARMER ARRANGEMENT FOR HEATING A FLUID IN A FLUID TUBE

(71) Applicant: OSAA INNOVATION APS, Hillerød (DK)

(72) Inventor: Ahmed Abdullah Hessam, Hillerød (DK)

(21) Appl. No.: 19/667,749

(22) Filed: May 5, 2026

Related U.S. Application Data
(63) Continuation of application No. PCT/DK2025/050193, filed on Nov. 7, 2025.

(30) Foreign Application Priority Data
Nov. 7, 2024 (DK) PA 2024 30673
Jun. 2, 2025 (DK) PA 2025 30355

(51) Int. Cl. H05B 3/26

(52) U.S. Cl. CPC H05B 3/26; H05B 2203/021

(57) ABSTRACT

The application relates to a warmer arrangement for heating a fluid in a fluid tube of an IV system. The warmer arrangement including at least one edge portion a first housing part forming at least a part of a housing, wherein the first housing part includes at least one wall including a first wall and one or more edge portions of the at least one edge portions. The at least one edge portion includes at least one pair of openings. The warmer arrangement also includes a track guide structure including an elongate track guide, and a heating device including at least one heating body arranged between the track guide structure and the first wall.

### TECHNICAL FIELD

The present invention relates to a warmer arrangement for heating a fluid in a fluid tube. More specifically, the present invention relates to a warmer arrangement for heating a fluid in a fluid tube of an infusion system, such as an IV system.

### BACKGROUND ART

The use of intravenous (IV) fluid administration is a critical component of medical care, widely applied in hospitals, emergency settings, and outpatient clinics to administer medications, blood products, and hydration to patients. Typically, IV fluids are stored at room temperature or in the fridge, which usually is lower than the preferred temperature of the human body around approximately 37° C. When large volumes of cold fluids are infused into a patient, it can lead to adverse effects such as hypothermia, which can complicate clinical outcomes and prolong recovery time.

Hypothermia induced by IV fluid administration is particularly concerning in trauma care, emergency surgery, and intensive care settings, where patients are often exposed to cold environments or have reduced body temperature due to blood loss. In such cases, maintaining the preferred temperature of the human body is critical for preventing physiological complications, including cardiac dysfunction and coagulation impairment.

Thus, there is a need for a solution, which can improve IV treatments by at least providing a portable solution that can heat fluid efficiently.

### DISCLOSURE OF INVENTION

An objective of the invention is to provide a warmer arrangement for heating a fluid flowing through a tube, which warmer arrangement relatively fast and efficient can heat the fluid to a selected temperature.

In an embodiment, it is an objective of the invention to provide a warmer arrangement for heating a fluid flowing through a tube, which warmer arrangement is portable and thereby can heat the fluid in fluid tube while being attached to a user or a device.

In an embodiment, it is an objective to provide a cost-efficient warmer arrangement, which is durable and compatible with commercially available standard IV tubes.

In an embodiment, it is an objective of the invention to provide a warmer arrangement, which precisely can heat a fluid to around the preferred human body temperature relatively fast, to ensure a safe IV treatment to a patient.

These and other objectives have been solved by the invention or embodiments thereof as defined in the claims and/or as described herein below.

It has been found that the invention or embodiments thereof have a number of additional advantages, which will be clear to the skilled person from the following description and embodiments.

Throughout the description and claims, the singular encompasses the plural, and the plural encompasses the singular unless otherwise specified or required by the context.

It should be emphasized that the term “comprises/comprising” when used herein is to be interpreted as an open term, i.e. it should be taken to specify the presence of specifically stated feature(s), such as element(s), unit(s), integer(s), step(s) component(s) and combination(s) thereof, but does not preclude the presence or addition of one or more other stated features.

Reference made to “some embodiments” or “an embodiment” means that a particular feature, structure, or characteristic described in connection with such embodiment(s) is included in at least one embodiment of the subject matter disclosed. Thus, the appearance of the phrases “in some embodiments” or “in an embodiment” in various places throughout the specification is not necessarily referring to the same embodiment(s). Further, the skilled person will understand that particular features, structures, or characteristics may be combined in any suitable manner within the scope of the invention as defined by the claims.

The term “substantially” should herein be taken to mean that ordinary product variances and tolerances are comprised.

All features of the invention and embodiments of the invention as described herein including ranges and preferred ranges may be combined in various ways within the scope of the invention, unless there are specific reasons not to combine such features.

The invention comprises a warmer arrangement suitable for heating a fluid flowing through a tube.

The warmer arrangement comprises at least one edge portion.

The warmer arrangement comprises a first housing part forming at least a part of a housing. The first housing part comprises at least one wall comprising a first wall and one or more edge portions of the at least one edge portion attached to and/or integrated with the first wall. Advantageously, the one or more edge portions of the first housing part at least partially surround the first wall to form an edge structure of the at least one wall.

In an embodiment, the one or more edge portions of the first housing part extends along at least a part of a periphery of the first wall, wherein the one or more edge portions are attached to or integrated with first wall at said periphery.

The at least one edge portion, such as the one or more edge portions of the first housing part comprises at least one pair of openings. The openings of the pair of openings may be formed between adjacent edge portions of the at least one edge portion.

In an embodiment, the at least one pair of openings at least partially through the edge structure comprises that the at least one edge portion comprises said at least one pair of openings.

The warmer arrangement comprises a track guide structure comprising an elongate track guide.

In an embodiment, the elongate track guide is channel shaped and extends at least partially from a first opening of the least one pair of openings to a second opening of the at least one pair of openings.

Advantageously, the elongate track guide has a length and comprises a channel extending along at least a part of a length of the elongate track guide, and wherein the elongate track guide extends at least partially from a first opening of the least one pair of openings to a second opening of the at least one pair of openings.

The warmer arrangement comprises a heating device comprising at least one heating body arranged between the track guide structure and the first wall.

The inventors have found that the elongate track guide and the heating device together provide a fast and efficient heating of fluid located and/or flowing inside a tube when the tube is located in the elongate track guide, such as in the channel of the elongate track guide.

In an embodiment, the track guide structure is abutting at least one of the first and the second openings of the at least one pair of openings.

It has been found that when the track guide structure is abutting at least one of the first and second openings the elongate track guide will be aligned with at least one of the first and second openings, which allow a tube to be arranged in the elongate track guide more easily while providing a faster heating of the fluid in the tube.

In an embodiment, the at least one wall comprises a second wall. The first wall and the second wall may form two opposite walls of the first housing part. The second wall may be located on an opposite site of the heating device, so that the first wall is located on a first side of the heating device and the second wall located on an opposite side of the heating device. The second wall may be located at the same side of the heating device as the first wall, and preferably, an insulating material, such as an insulating layer may be located between the first and second wall.

Preferably, the one or more edge portions of the first housing part are attached to each or attached to respective of the two opposite walls, preferably detachably attached to at least one of the two opposite walls. When detachably attached, the at least one of the two opposite walls may be removed and attached repeatedly to the one or more edge portions of the first housing part.

At least a portion of the at least one wall preferably the first wall comprises a curved outer shape, which is adapted to fit a shape of the skin or a body structure of a user.

It has been found by the inventors, that the warmer arrangement ensures a fast heating of the fluid in the fluid tube while being portable, as the structure of the warmer arrangement allow heat from the heating body to dissipate effectively to the elongate track guide while preventing heat from dissipating the housing part.

In an embodiment, the first housing part comprises four edge portions, which are pairwise located opposite to each other and wherein at least one of the pairwise edge portions are mutually parallel. The first housing part may comprise two or more edge portions based on the geometry of the first housing part.

The number of edges defining the geometry of the circumference of the first housing part is typically used when selecting the number of edge portions, as an edge portion may be defined as extending between two edges. A circular geometry of the circumference of the first housing part may not have any edges and thereby comprise one edge portion, whereas a square or rectangular circumference geometry of the first housing part may have four edges and thereby comprise four edge portions. The geometry of the circumference of the first housing part may be defined as the shape of an external and/or internal surface of the edge portions.

In an embodiment, the first opening and the second opening of the at least one pair of openings are arranged in a single plane parallel to a plane of the at least one wall. Preferably, a center point of each of the first and second openings are located in the single plane.

Advantageously, at least one of the first wall and the second wall of the first housing comprises a plane surface defining a plane of the at least one wall and wherein the first opening and the second opening of the at least one pair of openings are arranged in a single plane preferably parallel to the plane of the at least one wall.

In an embodiment, the plane of the at least one wall is defined as a plane comprising a flat rigid surface when the first housing portion is placed onto the flat rigid surface with the first wall in contact with the flat rigid surface.

In an embodiment, each of the openings of the at least one pair of openings are arranged with a minimum opening distance from the first wall, preferably the minimum opening distance of the first opening is different from the minimum opening distance of the second opening. The minimum opening distance may be measured as the distance from the first wall to a part of each of the first and second openings being closest to the first wall.

In an embodiment, the at least one pair of openings is arranged adjoining or neighboring in the at least one edge portion, such as the one or more edge openings of the first housing par, such as with a distance between each of the first and second openings in the at least one pair of openings being less than 1 cm, such as less than 5 mm, such as less than 2 mm.

In an embodiment, the first opening and the second opening of the at least one pair of openings are located immediately adjacent to each other or integrated with each other.

Preferably, each of the openings has a diameter from 2 mm to 8 mm, such as from 3 mm to 6 mm.

In an embodiment, the at least one edge portion, such as the one or more edge portions of the first housing part comprises two pairs of openings, preferably the two pairs of openings comprise openings mutually arranged opposite to each other.

In an embodiment, for at least one opening of the at least one pair of openings, the at least one edge portion, such as the one or more edge portions of the first housing part comprises a sliding door element, and wherein the sliding door element is adapted to move between an open configuration where the at least one opening is substantially open, such as fully or partially open and a closed configuration where the at least one opening is substantially closed, such as fully closed.

Preferably the sliding door elements is movable between the open configuration and the closed configuration by applying a force to an edge of the sliding door element.

In an embodiment, the sliding door element is arranged in the at least one opening.

Optionally, the at least one edge portion, such as the one or more edge portions of the first housing part comprises a sliding door element for a plurality of openings of the at least one pair of openings. Typically, each of the sliding door elements are arranged in one or more cavities of the at least one edge portion, such as the one or more edge portions of the first housing part, and wherein the one or more cavities comprises a cavity axis which is at least partially transverse to the at least one wall, and wherein the sliding door elements moves along the cavity axis.

Preferably, each of the sliding door elements is arranged in the at least one edge portion and is adapted to move lengthwise in one or more cavities of, or formed by the at least one edge portions, and wherein the one or more cavities comprises a cavity axis which, preferably is at least partially parallel to the plane of the at least one wall, and wherein the sliding door elements are adapted to move along the cavity axis.

The inventors have found that sliding door elements prevent heat from leaving the interior of the housing and thereby provides a more efficient heating of the fluid in the fluid tube, as less power is required to heat the fluid to a selected temperature.

In an embodiment, the track guide structure forms part of or is integrated with one of the first wall and the second wall of the first housing part. Preferably, the first and or second wall is detachably attached when the track guide structure forms part of is integrated with one of the walls.

The track guide structure is or is adapted for being located within the one or more edge portions of the first housing part and has a first surface and a second surface. The elongate track guide is recessed in the first surface, which allows a tube to be arranged within the recess at least partly. Advantageously, the channel of the elongate track guide is provided by the recess.

In an embodiment, the first surface of the track guide structure comprises one or more protruding elements protruding from the first surface of the track guide structure. The one or more protruding elements. Advantageously, the one or more protruding elements are arranged at the at least one bend to provide a simpler and more effective feed of the tube into the channel.

In an embodiment, the one or more protruding elements forms part of the elongate track guide, preferably the one or more protruding elements forms part of a wall of the channel.

Preferably, the one or more protruding elements are arranged at the at least one bend and adapted to at least temporarily retain the tube in the channel of the elongate track guide.

In an embodiment, the one or more protruding elements extends along a section of the channel of the elongate track guide, preferably extending at least 0.5 cm along the channel of the elongate track guide, more preferably at least 1 cm.

In one embodiment, the recessed elongate track guide protrudes at least partly from the second surface of the track guide structure.

In an embodiment, the track guide structure is configured to be detachably attached to one or more attachment elements, such as via a snap lock. The one or more attachment elements secure the track guide structure within the one or more edge portions of the first housing part. Typically, the one or more attachment elements are arranged at an inwardly facing surface of the one or more edge portions of the first housing part and/or at an inwardly facing surface of the first wall or the second wall.

“Inwardly facing” is herein used to define an orientation being towards the interior of a housing formed by the first housing part and a cover, such as a second housing part e.g. as described below, wherein the cover is covering the track guide structure.

The elongate track guide is adapted to fit a tube, such as a tube having an outer diameter of from 0.5 mm – 2 cm, such as from 0.8 mm to 1 cm, such as from 1 – 8 mm.

In an embodiment, the elongate track guide forms a cradle and/or a U-shaped profile, preferably suitable for supporting a tube.

In an embodiment, the elongate track guide, has a profile with a radius of 0.2 mm to 4 cm, such as from 0.5 mm – 2 cm, such as from 1 mm - 1 cm, preferably the elongate track guide, has a profile corresponding to an outer radius of a tube, such as a tube with an outer diameter of from 0.5 mm – 2 cm, such as from 0.8 mm to 1 cm, such as from 1 – 8 mm.

In an embodiment, the channel of the elongate track guide, has a profile with a radius of 0.2 mm to 4 cm, such as from 0.5 mm – 2 cm, such as from 1 mm - 1 cm, preferably the elongate track guide, has a profile corresponding to an outer radius of a tube, such as a tube with an outer diameter of from 0.5 mm – 2 cm, such as from 0.8 mm to 1 cm, such as from 1 – 8 mm.

In an embodiment, the elongate track guide has a width from about 0.1 mm to about 5 mm, such as from about 1 mm to about 3 mm, and/or a depth from about 0.1 mm to about 7 mm such as from 1 mm to about 5 mm, which allow standard IV tubes to fit in the elongate track guide.

The width may be determined as the cross-sectional maximal width of the recess.

The depth may be determined as the distance between a pair of parallel planes comprising a first plane comprising edges of the recess and a second plane comprising a bottom of the recess,

Preferably, the elongate track guide extends from a first end of the track guide structure to an opposite second end of the track guide structure.

In an embodiment, the elongate track guide comprises a fastening member configured to at least partially retain a tube arranged in the elongate track guide. The fastening member may protrude from an inner surface of the elongate track guide, such as from an inner surface of the channel of the elongate track guide, and preferably towards a center plane of the elongate track guide extending along a center axis of the elongate track guide.

Preferably, the track guide structure is attached, preferably detachably, to the one or more edge portions of the first housing part and/or to the heating device, and/or to the first housing part, such as the first wall and/or the second wall of the first housing part, preferably to the at least one wall. Preferably, the track guide structure is attached using a quick release mechanism.

Preferably, the heating body is configured for heating the elongate track guide to a temperature in the range of 35 to 43° C.

The heating body is located within the housing part and extends along the at least one wall. In an embodiment, the heating body comprise a plurality of heating bodies preferably connected to each other. The plurality of heating bodies may be located along the elongate track guide to direct the heat released from the heating bodies towards the elongate track guide.

In an embodiment, at least two of the plurality of heating bodies are configured for heating a part of the elongate track guide to different temperatures, such as wherein a first heating body is configured for heating the respective part of the elongate track guide to a temperature of 40° C and a second heating body is configured for heating the respective part of the elongate track guide to a temperature of 37° C.

It has been found that embodiments comprising a plurality of heating bodies are surprisingly effective for heating a fluid in a tube fast, as the temperature of each of the heating bodies may be controlled independently of the other heating bodies, while preventing the temperature of the fluid from exceeding a selected limit, usually around 37° C. Hence, in a desired embodiment, the heating device comprises a plurality of heating bodies.

In an embodiment, the warmer arrangement comprises at least one temperature presetting for heating the elongate track guide to a temperature in the range of 35 to 43° C, such as comprising a presetting for heating the elongate track guide to a temperature of about 37° C and optionally as comprising a presetting for heating the elongate track guide to a temperature of about 40° C.

Advantageously, the heating body is adapted to abut the elongate track guide structure at least partially, which allows heat to transfer from the heating body towards the elongate track guide. Preferably, the heating body is adapted to abut at least a portion of the elongate track guide protruding from the second surface of the track guide structure.

In an embodiment, the heating body is configured for heating the track guide structure or the elongate track guide by conduction.

Preferably, the heating body is a conductive heating body, and the track guide structure is a conductive track guide structure, each comprising a conductive material.

The conductive material may have a heat conductivity of at least about 25 W/(m*K), such as at least about 50 W/(m*K), such as at least about 75 W/(m*K), such as at least about 100 W/(m*K). The conductive material typically comprises iron, silver, copper, steel, gold, aluminum, or any combinations comprising one or more of these.

In an embodiment, the heating body is configured for heating the track guide structure or the elongate track guide by at least one of conduction and convection.

In an embodiment, the warmer arrangement comprises a blower or propeller located to circulate fluid in contact with the heating body and preferably to circulate fluid in contact with the second surface of the track guide structure.

In an embodiment, the heating body comprises an inductive coil adapted to provide an electrical field for heating the conductive track guide structure.

Advantageously, the heating device comprises a controller adapted for controlling the heating device. Preferably, the controller is adapted for automatic or remote controlling the temperature of the heating device.

Preferably, the controller comprises a frequency regulator adapted to regulate the intensity and/or frequency of the electrical field to control a rate of heat released from the heating body and the current temperature of the conductive track guide structure.

The heating device comprises a power source configured to power the heating body. The power source may be electrically connected to the heating device and may be located within the first housing part, within the housing or outside the housing. Preferably, the heating device is rechargeable.

In an embodiment, the heating device comprises at least one temperature sensor associated with the heating body for controlling the temperature of the heating body. The heating device may comprise a plurality of temperature sensors for providing a precise temperature across the whole surface of the heating body.

In an embodiment, the heating body comprises an infrared emitting element configured to emit infrared radiation.

Preferably, the controller comprises an emitter regulator adapted to regulate the intensity and/or wavelength of the radiation from the infrared emitting element and the current temperature of the track guide structure.

In an embodiment, the heating body is adapted for heating the elongate track guide of the track guide structure up to a temperature not exceeding 50° C.

In an embodiment, the heating body is adapted for cooling the elongate track guide of track guide structure down to a temperature not exceeding 40° C, such as not exceeding 39° C, such as not exceeding a temperature of 38° C.

In an embodiment, the heating body comprises a plurality of heating bodies, and wherein at least one of the heating bodies is configures for cooling the track guide structure.

In an embodiment, the arrangement comprises a set of housing parts comprising the first housing part and a second housing part forming a housing. The set of housing parts are preferably adapted for being temporally or permanently connected to each other, preferably rotatably connected to each other through a rotating device, such as a hinge, and at least partially retained connected by a at least one locking element, such as a slide locking mechanism, a snap-fit joint mechanism, or a latch mechanism.

In an embodiment, wherein the arrangement comprises a set of housing parts comprising said first housing part and the second housing part, the set of housing parts are adapted for being temporally or permanently connected to each other and at least partially retained connected by at least one locking element.

The second housing part advantageously comprises at least one second housing part edge portion of the at least one edge portion.

The least one second housing part edge portion may correspond to the one or more edge portions of the first housing part.

The second housing part advantageously comprises at least one second housing part wall, which may correspond to the first wall of the first housing part, e.g. by mirroring the shape of the first wall of the first housing part.

The first wall of the first housing part, the one or more edge portions of the first housing part, the at least one second housing part edge portion and the at least one second housing part wall may preferably together form the outer walls, and thereby the outer surface of the warmer arrangement.

Advantageously the set of housing parts are adapted for being connected together by abutting the at least one second housing part edge portion with the one or more edge portions of the first housing part.

The set of housing parts may be rotatably connected to each other through a rotating device, such as a hinge, e.g. having a clamp shell structure.

Preferably, the set of housing parts are adapted for being connected together by abutting at least a pair of the at least one edge portions of each of the respective first and second housing parts.

Advantageously, the elongate track guide extends in a plane parallel to the at least one wall.

In an embodiment, the elongate track guide comprises a meandering extension. The meandering extension may comprise at least one bend and/or one or more folds and/or an accordion folded extension.

Advantageously, the elongate track guide extends in a meandering path, preferably comprising at least one bend, such as a plurality of bends, such as an accordion bended path.

The extension of the elongate track guide, also referred to as the configuration of the elongate track guide may be selected based on characteristics of the IV system used and the characteristics of the fluid, such as initial fluid temperature. For example, when the initial fluid temperature is below 10° C, the length of the elongate track guide may be longer than when the initial fluid temperature is 20° C, as the tube must be heated for a longer period or at a higher temperature. The length may be reflected by the meandering shape of the elongate track guide.

Preferably, the arrangement comprises a monitor sensor and a display for monitoring and displaying least one condition of a fluid tube arranged in the elongate track guide, the condition preferably comprises a temperature of a fluid in the tube and/or at least one dispensing condition, such as flow rate or dispensing status.

In an embodiment, the display is configured for displaying at least one condition of the heating device and/or at least one condition of a fluid tube arranged in the elongate track guide. Alternatively, The arrangement may comprise one or more electric powered signal elements, the signal elements are preferably arranged on an outer surface of the at least one wall of the first housing part, an outer surface of the at least one second housing part wall and/or an outer surface of the one or more edge portions of the first housing part and/or an outer surface of the at least one second housing part edge portion. The signal elements are conveniently configured for displaying at least one condition of the heating device and/or at least one condition of a fluid tube arranged in the elongate track guide, such as by illuminating different color codes.

Preferably, the warmer arrangement comprises a fixation element configured to at least partially fixate the arrangement to a limb of a user or an external device, such as an IV-pole.

In an embodiment, the track guide structure comprises a support plate supporting and/or integrated with the elongate track guide.

Conveniently, at least a portion of the second wall of the first housing part form part of or constitutes the supporting plate.

The support plate has the function of supporting the elongate track guide, to provide that the elongate track guide is not deformed if or when subjected to a force towards the heating device, e.g. as explained further below.

In an embodiment, the elongate track guide is detachable fixed to the support plate.

Advantageously, the elongate track guide is integrated with the support plate or permanently fixed to the support plate. Thereby the support plate may form part of the track guide structure.

In an embodiment, the track guide structure consists of the track guide structure and the elongate track guide.

The support plate may conveniently have a first side with a first surface, wherein at least a first portion of the first surface of the support plate is in physical contact with the at least one heating body of the heating device.

In an embodiment, the support plate has a first surface with a first surface path, the elongate track guide extends in a track path, and wherein the first surface path is in conformation with the track path. Thereby the first surface path may correspond to the track path.

Preferably the first portion of the first surface of the support plate in physical contact with the at least one heating body comprises the first surface path of the support plate. Thereby, the heat transfer to a fluid in a tube located in the elongate track guide, such as in the channel of the elongate track guide may be increased.

By providing that the first portion of the first surface of the support plate in physical contact with the at least one heating body comprises said first surface path a valuable improvement of the warmer arrangement may be obtained.

To further ensuring a fast temperature regulation of a fluid in a tube located in the elongate track guide e.g. the channel of the elongate track guide it is desired that a second portion of the first surface outside the first portion of the first surface of the support plate is without physical contact to the heating body.

Advantageously, the second portion of the first surface outside the first portion of the first surface of the support structure is thermally isolated from the heating body. For example, a thermally insulating material may be located between the second portion of the first surface and the heating body.

Thereby, the amount of heat that may dissipate from the heating device without being used for heating a fluid in a tube located in the elongate track guide may be effectively reduced.

In an embodiment, a thermally insulating material is located between the support plate and the first wall of the first housing part. Thereby, providing an additional barrier against undesired loss of heat from the heating device. The insulating material may be located between the support plate and the first wall of the first housing part may e.g. be in one or more layers of insulation to effectively prevent heat to dissipate via the first wall, which may conveniently be adapted to be in contact with a patient’s skin.

Advantageously the thermally insulating material has a thermal conductivity of 0.1 W/m*k or less, such as thermal conductivity of 0.05 W/m*k or less, such as thermal conductivity of 0.04 W/m*k or less, such as thermal conductivity of 0.035 W/m*k or less, such as thermal conductivity of 0.02 W/m*k or less.

Examples of thermally insulating material comprises glass fibers, glass wool closed-cell foam, and polyurethane (PU) foam.

In an embodiment, the support plate has a second side and the elongate track guide may beneficially be formed by an elongate elevated structure protruding from a second side of the support plate.

Preferable the elevated structure protruding at a second side of the support plate form part of or constitutes the elongate track guide.

In an embodiment, the channel of the elongate track guide is at least partially formed by the elongate elevated structure.

Beneficially, the elongate elevated structure comprises the recess providing the channel.

The elongate track guide, such as the elongate elevated structure may preferably have a relatively high thermal conductivity to provide a desired high temperature regulation of a fluid in a tube located in the elongate track guide e.g. the channel of the elongate track guide.

In an embodiment, at least the elongate elevated structure comprises or consists of at least one material having a heat conductivity of at least 25 W/(m*K), such as at least 50 W/(m*K), such as at least 75 W/(m*K), such as at least 100 W/(m*K), preferably comprising iron, silver, copper, steel, gold, aluminum, or any combinations comprising one or more of these.

In an embodiment, the warmer arrangement comprises a track guide lid cover. The track guide structure together with the track guide lid cover may preferably form an elongate encasing path, suitable for encasing a length of the tube.

The track guide lid cover is preferably located or adapted to be located between the first housing part and the second housing part or advantageously the track guide lid cover forms part of the second housing part. By ensuring that the track guide lid cover forms part of the second housing part the warmer arrangement may be simpler to use since the number of element to handle may be kept low, i.e. the track guide lid cover need not be handled individually.

In an embodiment, the channel has a semi-closed cross-section perpendicular to a center axis of the channel with an elongate opening into the channel extending along the length of the elongate track guide. The term “semi closed” means herein that the elongate opening into the channel extending along the length of the elongate track guide is relatively narrow, e.g. wherein the maximal width of the elongate opening into the channel is from 5 % to 40 % of the maximal width of the channel seen in the cross-section perpendicular to the center axis of the channel, preferably the maximal width of the elongate opening into the channel is from 10 % to 30 %, such as from 15 % to 25 % of the maximal width of the channel.

Thereby it becomes much simpler to feed the length section of the tube into the channel, in particular where the elongate track guide and thereby the channel has several, such as two or more bends or folds, because the risk that portions of the length section of the tube unintentionally slips out of the channel is highly reduced or even fully avoided, while it still remain simple to feed the length section of the tube into the channel and to withdraw the length section of the tube from the channel e.g. in fully or partly empty condition.

After the tube length section has been fed into the channel via the narrow elongate opening into the channel, the track guide lid cover may e.g. be applied to fill out the elongate opening. The fluid adapted to be heated in the warmer arrangement may be applied to flow in the tube length section after the tube length section has been fed into the channel and before or preferably after the track guide lid cover has been applied to fill out the elongate opening.

The track guide lid cover may therefore advantageously be fixed to or integrated with the second housing part.

Advantageously, the first housing part and the second housing part of the set of housing parts are adapted to be locked to each other and temporally held in locked position during the heating of a fluid flowing through a tube with a tube length section located in the elongate track guide, such as located in the channel of the elongate track guide.

In a desired embodiment, the track guide lid cover is adapted to be pressed towards the track guide and/or a tube length section located in the elongate track guide when the first housing part and the second housing part of the set of housing parts are locked to each other. The pressure applied to the track guide lid cover, may be applied by mounting the second housing part to the first housing part, e.g. by locating the second housing part above the first housing part with the track guide lid cover facing towards the elongate track guide, preferably comprising a length section of the tube located in the elongate track guide, preferably in the channel of the elongate track guide, and fixing the second housing part to the first housing part e.g. using the at least one locking element.

In an embodiment where the first housing part and the second housing part are connected via one or more hinge along first edge portions of respectively the first housing part and the second housing part, the first housing part and the second housing part may be rotated towards each other to provide that the track guide lid cover is pressing towards he track guide and/or a tube length section located in the elongate track guide and the first housing part and the second housing part may be locked to each other by the at least one locking element located at one or more second edge portions of the first housing part and/or of the second housing part.

In an embodiment, the elongate encasing path has a first diameter in a first plane orthogonal to the plane of the at least one wall and a second diameter in a second plane orthogonal to the first plane, wherein the first diameter is smaller than the second diameter, such as the first diameter is at least 2% smaller than the second diameter, such as the first diameter is at least 4% smaller than the second diameter, such as the first diameter is at least 6 % smaller than the second diameter, such as the first diameter is at least 8 % smaller than the second diameter.

Advantageously, the warmer arrangement comprises a thermally insulating material forming part of located between the second housing part and the track guide structure. Preferably, the track guide lid cover comprises or is of thermally insulating material.

In an embodiment, the first housing part and the second housing part are locked together or is adapted for being locked together to form an enclosing housing. Advantageously, the at least one locking element is adapted for holding the first housing part and the second housing part in a closed condition when the warmer arrangement is in operation.

Conveniently, the enclosing housing in closed condition is a closed housing, such as fully closed except for the at least one pair of openings. Thereby heat exchange between material, such as the heating device inside the enclosing housing and material, such as air and/or skin of a patient is kept at a minimum, e.g. heat dissipation and/or heat loss from the warmer arrangement is desirable low.

Advantageously the at least one locking element is adapted for holding the first housing part and the second housing part in tension towards each other when the first housing part and the second housing part are in closed condition.

In an embodiment, the at least one locking element is adapted for holding the first housing part and the second housing part in tension to exert a pressure acting onto the track guide structure at least when a length section is located in the elongate track guide, such as in the channel of the elongate track guide. Thereby the exerted pressure will cause that the tube length section will be pressed towards the elongate track guide, preferably towards the channel of the elongate track guide to increase the surface contact between the outer surface of the tube length section and the elongate track guide such as an inner surface of the channel of the elongate track guide.

In an embodiment, the warmer arrangement comprises an air detector. Preferably, the air detector comprises an acoustic sensing element adapted to detect air in the tube. Advantageously, the air detector can detect air in the tube when fluid is flowing in the tube. The air detector may use acoustic waves to identify changes in the fluid, such as air and/or air bubbles in the fluid.

Detection of air and/or air bubbles may cause the fluid flow device to constrict the fluid tube to prevent the detected air and/or air bubbles from being dispensed through tube.

The air detector is preferably in data connection with the controller. It has been found that using acoustic waves for detecting air and/or air bubbles in the fluid is very precise and reliable.

In an embodiment, the air detector is arranged adjacent to the fluid flow device. Preferably, the fluid flow device comprises the air detector.

In an embodiment, the warmer arrangement comprises a crystallized matter detector configured for detection crystals that may have formed in the fluid. If crystals are detected, the assist controller may conveniently cause the fluid flow device to constrict the fluid tube to prevent the detected crystals from being dispensed through the tube.

The controller may be configured for controlling at least one fluid flow device comprising one or more force acting elements at least partially in dependence of the air detector and/or at least partially in dependence of the air detector and/or at least partially in dependence of the crystallized matter detector. Preferably, the controller is configured for submitting an electrical signal to the at least one fluid flow device to provide the one of the one or more force acting elements to constricting the tube to terminate the fluid flow in the tube at least temporally, preferably the controller is configured for simultaneously providing an alarm.

In an embodiment, the track guide structure comprises a cartridge. The cartridge is preferably adapted to be removably inserted into the warmer arrangement, and the cartridge comprises a cartridge housing defining an internal chamber comprising the elongate track guide.

Preferably, the cartridge is made of a conductive material.

In one embodiment, the warmer arrangement comprises a plurality of cartridges, preferably the plurality of cartridges is arranged side by side. It has been found that a plurality of cartridges allow an efficient heating or cooling of a plurality of fluids without having to operate multiple warmer arrangement simultaneously.

Advantageously, the cartridge comprises at least two apertures for allowing fluid to enter and discharge from the cartridge. Preferably the at least two apertures are aligned with the at least one pair of openings that may provide a more efficient handling of the warmer arrangement when used, especially in a turbulent and/or a chaotic environment.

In an embodiment, the at least one of the at least two apertures is connected to a tube, preferably the tube comprises a tube connector and/or a drip chamber in the opposite end, preferably adapted to connect to another tube, such as an IV tube.

In an embodiment, the cartridge comprises a cartridge housing defining an internal chamber comprising the elongate track guide configured to direct fluid through the cartridge housing.

Advantageously, at least one of the at least two apertures may comprise a cartridge connector adapted for engagement with a tube. To provide a more efficient and easy-to-connect coupling of the cartridge to a tube, preferably an IV-tube of an IV system. It has been found that a cartridge connector is advantageous for providing a quick coupling to a tube and for preventing dirt and other unwanted foreign objects from entering the tube and the cartridge when coupled together.

In an embodiment, the cartridge is configured to prevent reuse by incorporating a breakable locking element when removed from the warmer arrangement. Advantageously, the breakable locking element destroys a portion of the cartridge or colors at least a part of the cartridge when broken. Preferably, a coloring agent is incorporated into the breakable locking element, and preferably the coloring agent is arranged in connection with the cartridge connector.

In an embodiment, the warmer arrangement comprises a fluid flow device, such as at least one fluid flow device for adjusting a fluid flow in the tube. The fluid flow device comprises one or more force acting elements, wherein the one or more force acting elements are configured for adjustably constricting the tube to adjust fluid flow.

It has been found that constricting the tube using force acting elements is a cost effective and efficient way of controlling the velocity and/or the flow rate and/or stopping of the fluid dispensed in the tube.

In an embodiment, the fluid flow device comprises a tube recess adapted to accommodate a tube, and wherein the one or more force acting elements are arranged at one or more sub-recesses in a tube recess wall having a passage into the tube recess.

The tube recess has a length along a tube recess center axis.

In an embodiment, the tube recess is defined by a tube recess wall. The tube recess wall advantageously comprises at least one sub-recess, wherein the at least one sub-recess comprises a recess passage into the tube recess.

In an embodiment, the one or more force acting elements of the fluid flow device comprises at least a pair of force acting elements arranged opposite to each other. Preferably, the fluid flow device comprises at least two pairs of force acting elements, and wherein each pair of force acting elements are independently operable. Advantageously, each of the force acting elements of a pair of force acting elements are opposite acting, preferably such that they are moving mirrored to each other.

Advantageously, the fluid flow device is arranged in the channel and/or forms part of the channel.

In an embodiment, the fluid flow device at least partly encloses a section of the channel.

Advantageously, the fluid flow device is adapted to stop the fluid flow in the tube by constricting the tube, preferably when receiving a signal from a controller. Also, the fluid flow device may be arranged at one end of the elongate track guide, preferably at one end of the channel.

The invention also comprises a warmer system for heating a fluid flowing through a tube. The warmer system comprises a warmer arrangement e.g. as described above and defined in the claims. The tube may advantageously be an infusion tube, such as an IV tube.

In an embodiment, the tube is an infusion tube, preferably connected to an infusion bag comprising an infusion fluid. Preferably, the tube form part of an infusion set comprising a drip chamber, optionally the warmer system comprises the infusion set. The infusion set may e.g. comprises a fluid assist arrangement as described in co-pending DK application PA 2024 30513.

In an embodiment, the tube comprises a temperature adjustment length section which may correspond to or be identical to the tube length section described above and which temperature adjustment length section is or is adapted to be applied in the elongate track guide, preferably in the channel of the elongate track guide.

Advantageously, the warmer arrangement comprises a track guide lid cover, wherein the track guide structure together with the track guide lid cover form an elongate encasing path, suitable for encasing a length of the tube, such as the temperature adjustment length section of the tube.

The track guide lid cover is beneficially adapted to be located between the first housing part and the second housing part. In an embodiment the track guide lid cover is located between the first housing part and the second housing part when the first housing part and the second housing part are locked to each other e.g. when the first housing part and the second housing part form an enclosing housing in the closed condition.

In an embodiment, the track guide lid cover forms part of the second housing part. Preferably, the track guide lid cover is fixed to or integrated with the second housing part e.g. as described above.

Beneficially, the first housing part and the second housing part of the set of housing parts are locked to each other or is adapted to be locked to each other and temporally held in locked position during the heating of a fluid flowing through a tube.

The track guide lid cover is preferably adapted to be pressed towards or is pressed towards a length of the tube, such as the temperature adjustment length section of the tube when the first housing part and the second housing part of the set of housing parts are locked to each other. Preferably the track guide lid exert a pressure onto the length of the tube, such as the temperature adjustment length section of the tube and preferably deforms the tube when the first housing part and the second housing part of the set of housing parts are locked to each other,

As explained above, it has been found that by pressing the tube length section towards the elongate track guide, such as towards the channel of the elongate track guide, a significant faster heating of the fluid flowing through the temperature adjustment length section may be obtained.

In an embodiment, the track guide lid cover is adapted to be pressed towards the track guide and/or towards the temperature adjustment length section and exert a pressure onto the tube, such as length of the tube, such as the temperature adjustment length section of the tube when the first housing part and the second housing part of the set of housing parts are locked to each other.

Preferably, the at least one locking element is adapted for holding the first housing part and the second housing part in tension when the first housing part and the second housing part are in closed condition The tension applied is advantageously sufficient to exert a pressure onto the tube, such as length of the tube, such as the temperature adjustment length section of the tube, wherein the exerted pressure may deform at least a portion of the length of the tube, such as the temperature adjustment length section of the tube.

Conveniently, the at least one locking element is configured for holding the first housing part and the second housing part in tension to exert a pressure acting onto the tube, such as length of the tube, such as the temperature adjustment length section of the tube located in the elongate track guide, such as in the channel of the elongate track guide, preferably to deform at least a portion of the length of the tube, such as the temperature adjustment length section of the tube.

In an embodiment, the at least one locking element is configured for holding the first housing part and the second housing part in tension, wherein the tension to exert a pressure acting onto the tube is sufficiently high to causing a deformation of the tube. The tension may preferably cause a pressure sufficiently high to increase a surface contact area between the inner surface of the channel and the outer surface of the tube, (such as the temperature adjustment length section of the tube), relative to a surface contact area between the inner surface of the channel and the outer surface of the tube without the deformation.

Preferably, the tension to exert a pressure acting onto the tube is causing a deformation of the tube, deforming the tube from having a circular cross-section to have an oval cross-section. Preferably, the tension to exert a pressure acting onto the tube is causing a deformation of the tube to have a maximal outer diameter and a minimal outer cross-sectional diameter, wherein the maximal outer cross-sectional diameter is larger than the minimal outer cross-sectional diameter, preferably with an aspect ratio of from 1,01 to 2, such as 1.05 to 1.5.

The outer diameter of the tube, and preferably at least the outer diameter of the temperature adjustment length section of the tube is selected in dependence of the channel, preferably such that the temperature adjustment length section may be applied in the temperature adjustment length section to have a good contact to the inner surface of the channel.

The channel of the elongate track guide comprises an inner surface, preferably having cross-sectional U-shape with a channel radius. The channel radius is determined as the radius of a circle contacting the inner surface of the channel at the bottom of the U bend of the channel.

Preferably, at least the temperature adjustment length section of the tube has an outer radius, which is from half of the channel radius to twice the channel radius.

More preferably, the at least the temperature adjustment length section of the tube has an outer radius, which is from 1.1 times larger to 1.5 times smaller than the channel radius.

The invention also comprises a kit of parts.

The kit of parts comprises an arrangement for heating a fluid flowing through a tube comprising at least one edge portion. The kit comprises a first housing part adapted for or forming a housing comprising at least one wall and one or more edge portions of the at least one edge portion attached in one end to the at least one wall. The at least one edge portion, such as the one or more edge portions of the first housing part comprising at least one pair of openings, and a heating device comprising at least one heating body arranged between the track guide structure and the at least one wall.

The kit of parts also comprises a plurality of track guide structures, each track guide structure comprising an elongate track guide with a different configuration adapted to fit a tube.

In an embodiment, the track guide structure is or is adapted for being arranged abutting the at least one edge portion providing a first end of the elongate track guide to be located abutting at least one of the first and the second of the at least one pair of openings.

Advantageously, each of the different configurations of the elongate track guides comprises a different elongate track guide width and/or depth, extension length, and/or extension shape, such as a meandering or an accordion folded extension.

In an embodiment, the arrangement for heating a fluid flowing through a tube is a warmer arrangement according to any one of the embodiments described above.

### BRIEF DESCRIPTIONS OF EMBODIMENTS AND EXAMPLES

In the following, the invention will be further illustrated by the description of a number of illustrative and non-limiting embodiments and examples of the present invention, with reference to the appended drawings.

The figures are schematic, are not drawn to scale, and may be simplified for clarity. Throughout, the same reference signs are used for identical or corresponding parts.

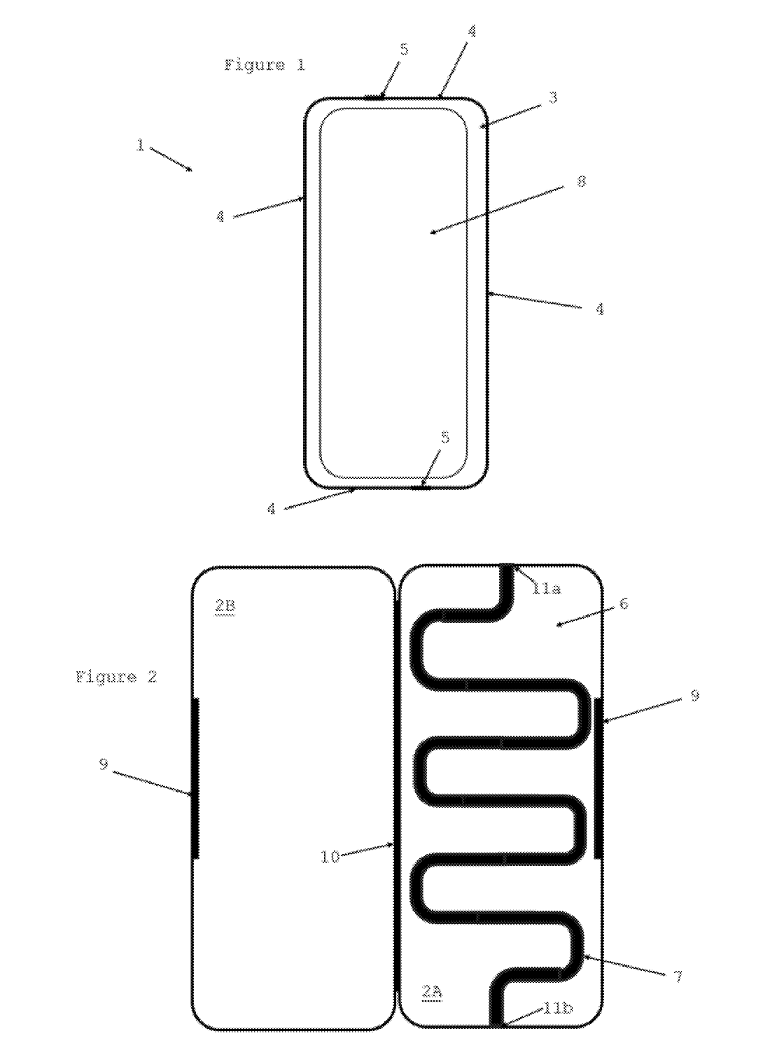

FIG. 1 illustrates an embodiment of the warmer arrangement of the invention.

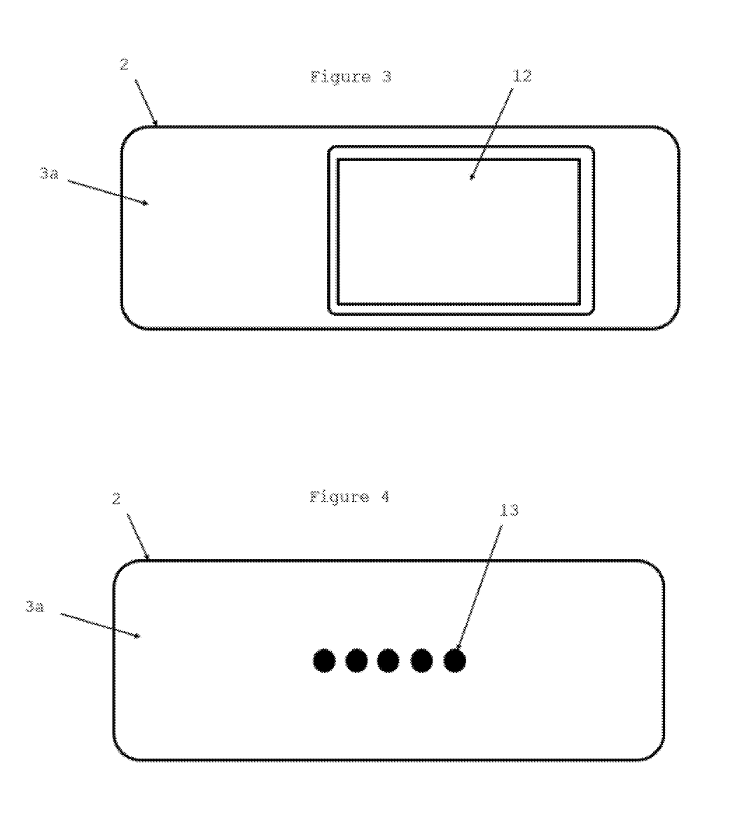

FIG. 2 illustrates an embodiment of the warmer arrangement of the invention comprising a set of housing parts.

FIG. 3 illustrates an embodiment of the warmer arrangement of the invention from a top view comprising a display.

FIG. 4 illustrates an embodiment of the warmer arrangement of the invention from a top view comprising signal elements.

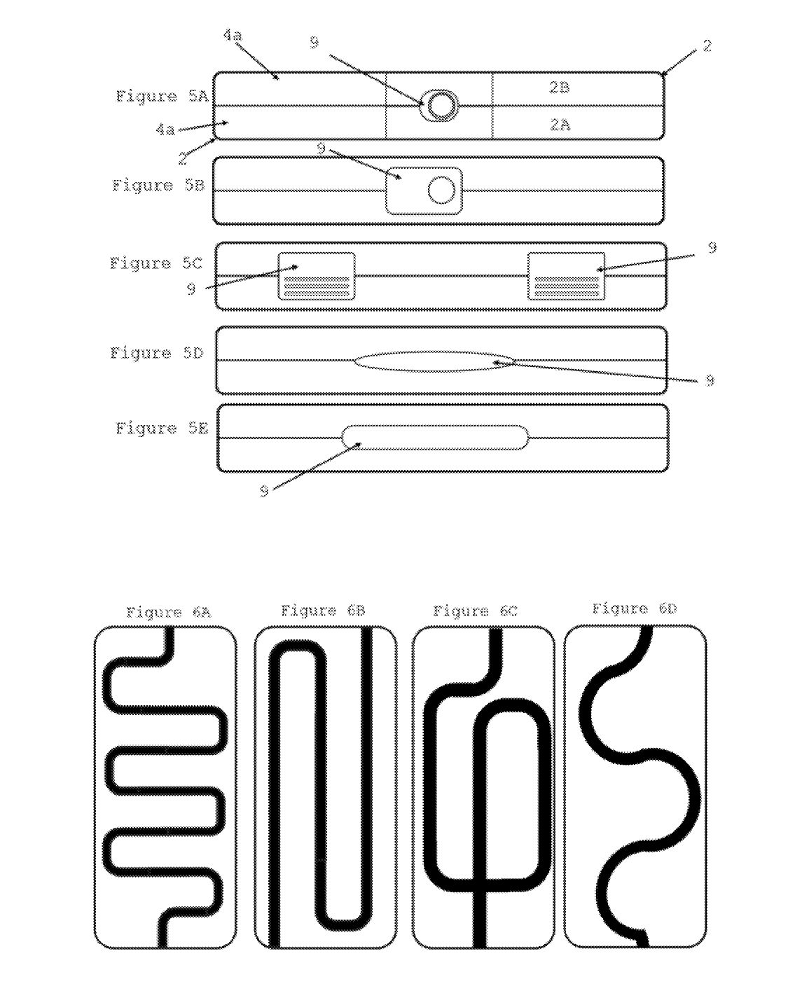

FIGS. 5A-5E illustrate different embodiments of the warmer arrangement of the invention from a side view comprising a set of housing parts connected.

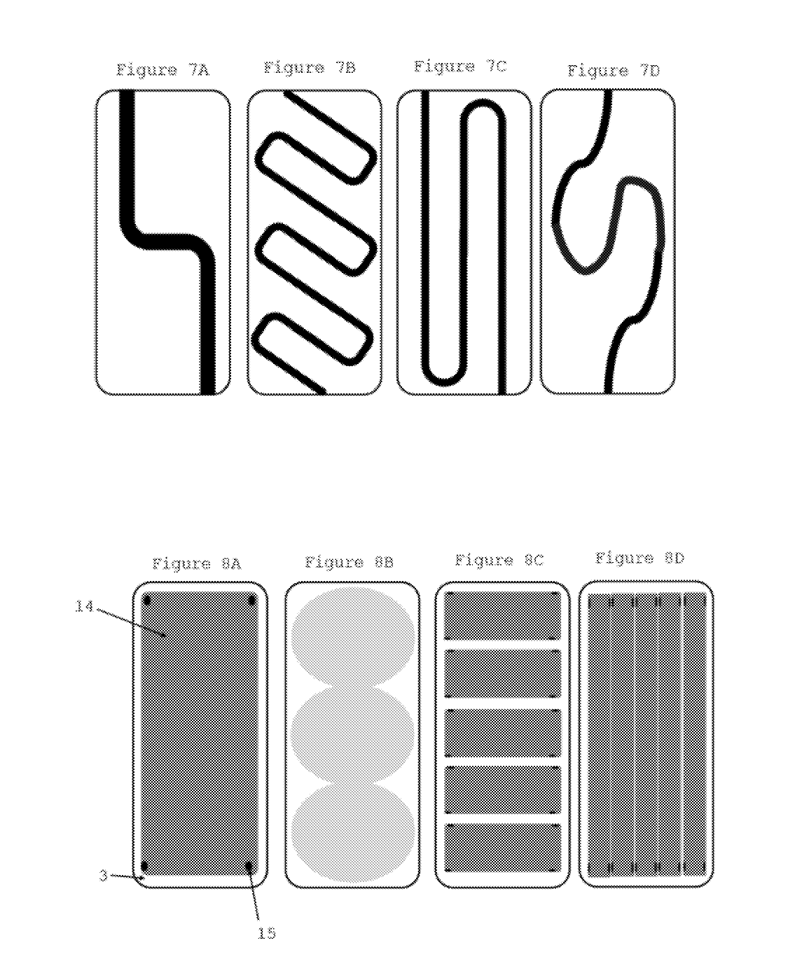

FIGS. 6A-6D and 7A-7D illustrate different embodiments of the elongate track guide structure comprising different configurations.

FIGS. 8A-8D illustrate different embodiments of the warmer arrangement of the invention comprising a heating device.

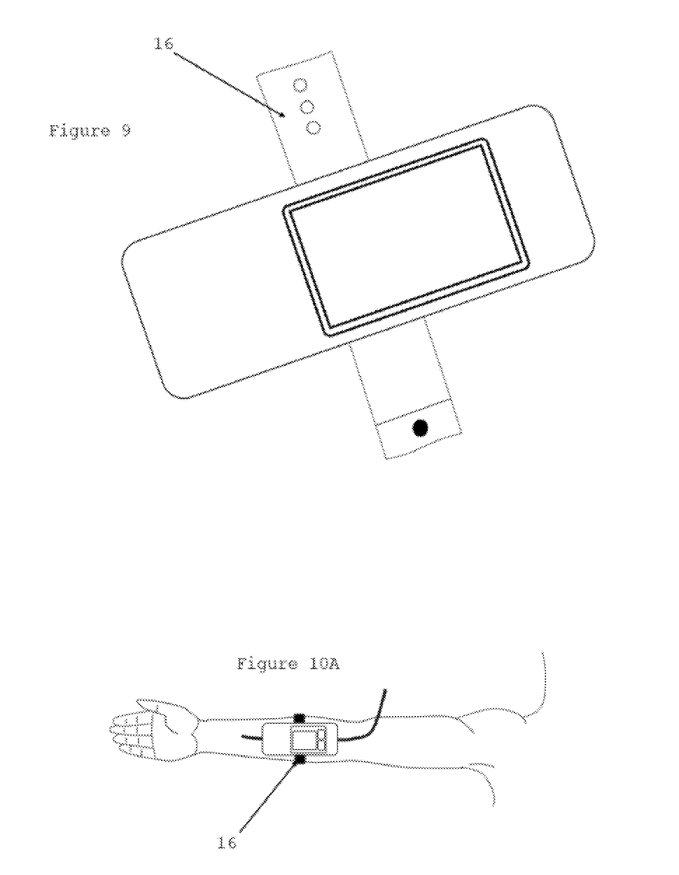

FIG. 9 illustrates an embodiment of the invention comprising a fixation element.

FIGS. 10A and 10B illustrate different embodiments of the invention attached to a limb of a user.

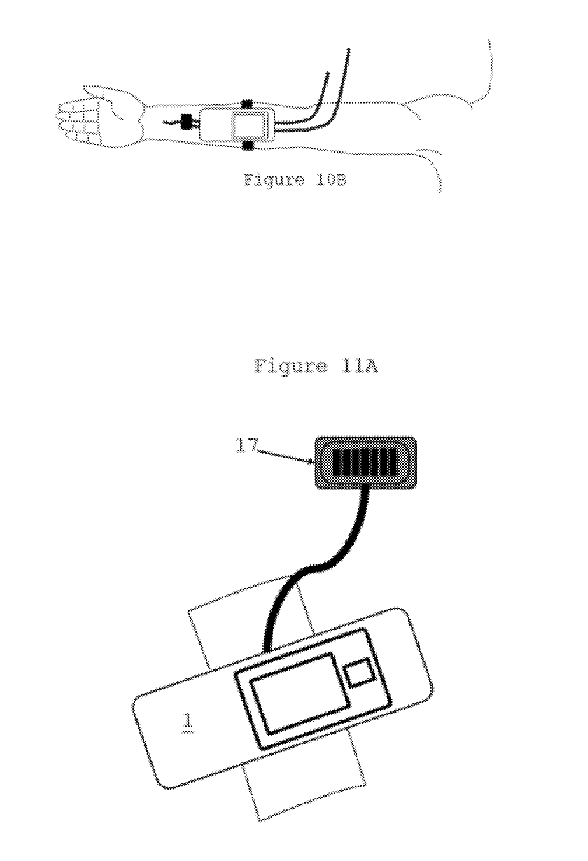

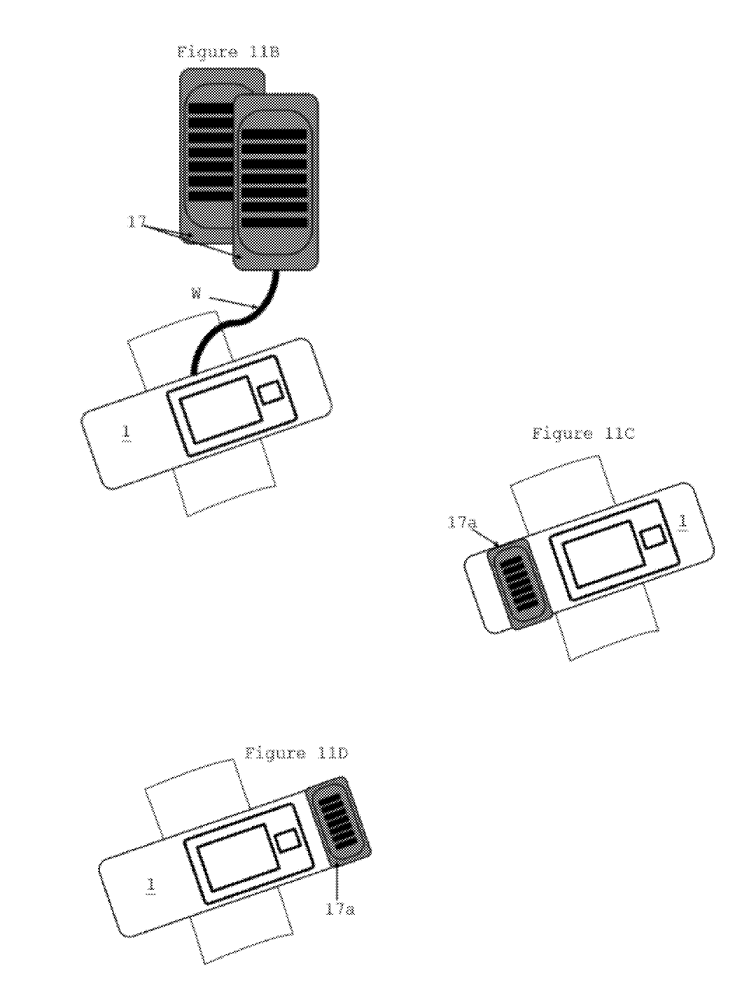

FIGS. 11A-11D illustrate different embodiments of the invention comprising a battery connected to the heating device.

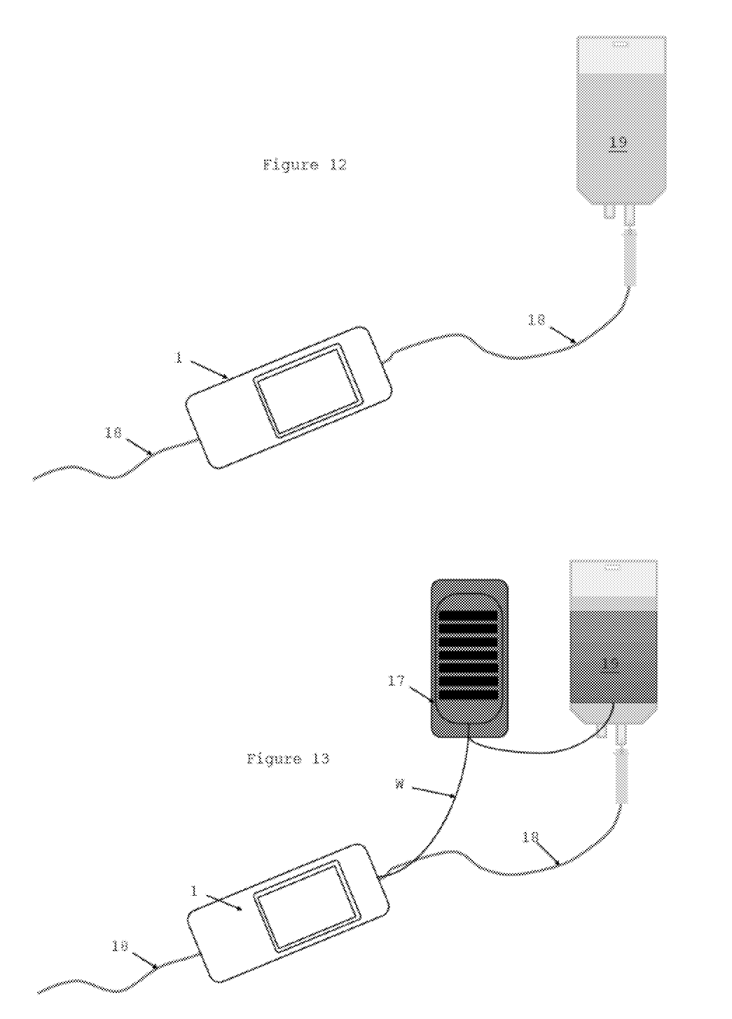

FIG. 12 illustrates an embodiment of the arrangement of the invention used with an IV system.

FIG. 13 illustrates an embodiment of the arrangement of the invention comprising a battery connected to an IV system.

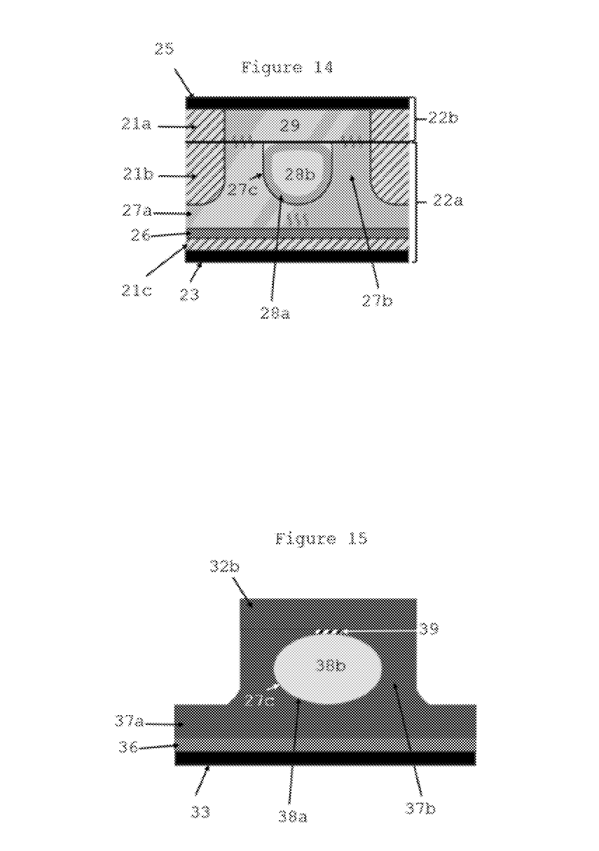

FIG. 14 is a cross sectional view of a part of a warmer arrangement of an embodiment of the invention comprising track guide structure comprising a support plate and an elongate track guide and a track lid cover.

FIG. 15 is a cross sectional view of a part of a warmer arrangement of an embodiment of the invention where the elongate track guide has a semi-closed cross-section with an elongate narrow opening into the channel of the elongate track guide.

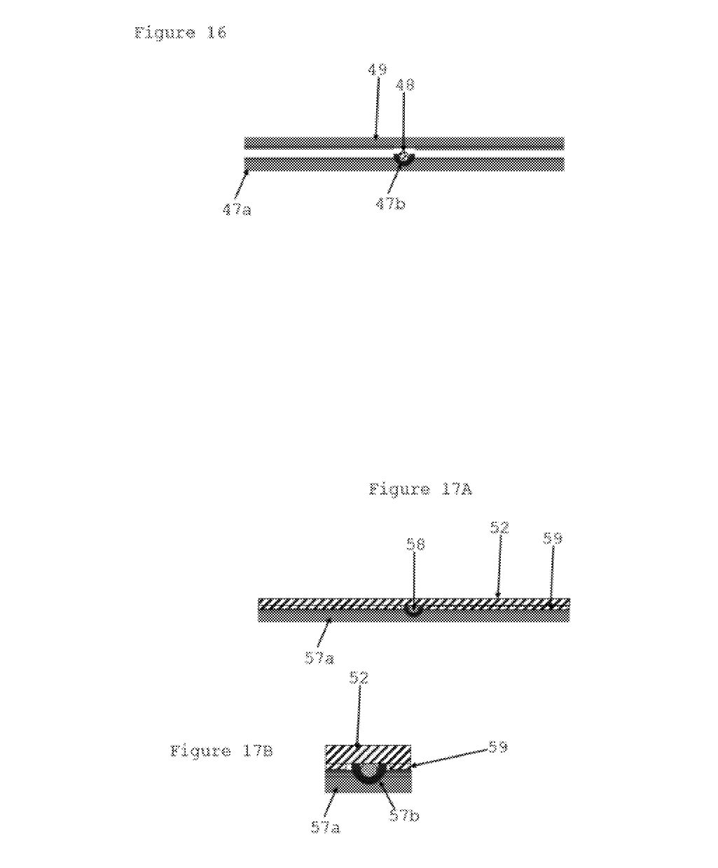

FIG. 16 is a cross sectional view of an embodiment of a track guide structure comprising a support plate and an elongate track guide and a tack lid cover adapted to cover the elongate track guide.

FIG. 17a is a cross sectional view of an embodiment a track guide structure with a tack lid cover applied to cover the elongate track guide to form an elongate encasing path.

FIG. 17b shows an enlarged portion of FIG. 17a.

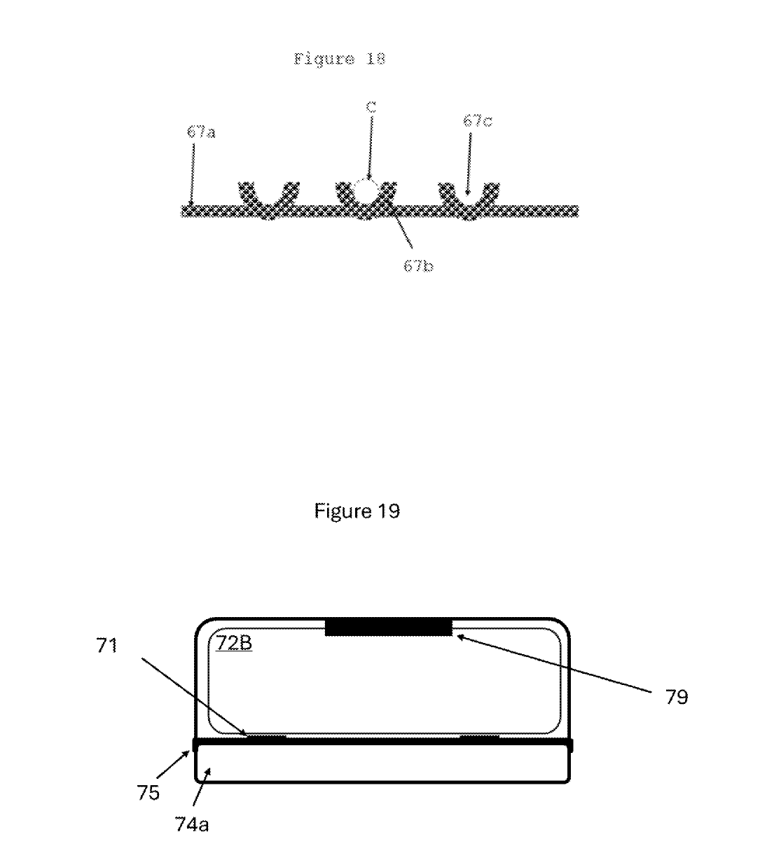

FIG. 18 illustrates a cross sectional view of a track guide structure comprising a support plate and an elongate track guide.

FIG. 19 illustrates an embodiment of the warmer arrangement from a side view comprising two protruding elements on the track guide structure.

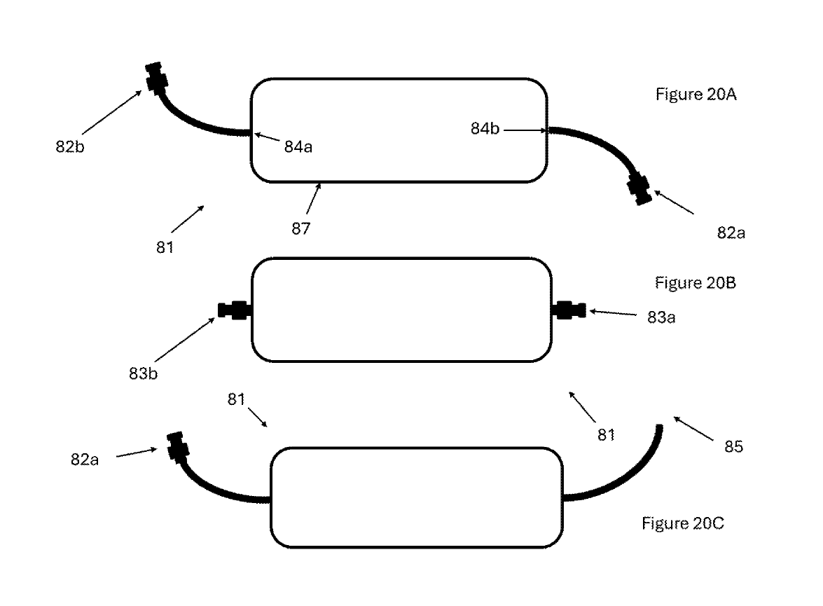

FIGS. 20A-20C illustrate different embodiments of a cartridge according to the invention.

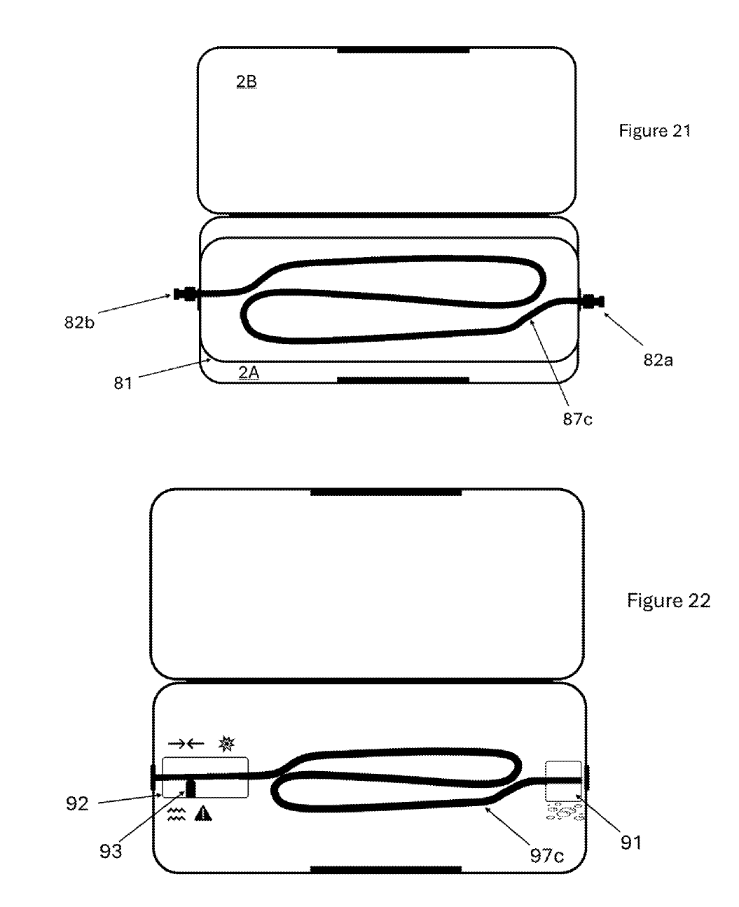

FIG. 21 illustrates a top view of an embodiment of the warmer arrangement of the invention comprising a cartridge.

FIG. 22 illustrates a top view of an embodiment of the warmer arrangement of the invention comprising a fluid flow device and an air detector.

With reference to FIG. 1, one embodiment of the invention will now be described. FIG. 1 illustrates a warmer arrangement 1 comprising a housing part, which could be the first housing part and/or the second housing part. The housing part comprises four edge portions 4 and a wall 3 surrounded by the four edge portions. The edge portions 4 extends around a circumference of the wall 3 and defines the interior of the housing part. Two of the four edge portions 4 being opposite to each other have each an opening 5 each allowing a tube to extend from the exterior of the housing part into the interior of the housing part through the opening at the respective edge portion.

In a version thereof, the housing part is the first housing part and the warmer arrangement comprises a heating device 8 located in the interior of the housing part. The heating device 8 is located onto on the wall 3, which is here the first wall of the first housing part. In the shown embodiment, the heating device has a shape similar to the shape of the wall 3. The skilled person will understand that the heating device may have other shapes, for example, the heating device may be shaped as shown in any of FIGS. 8A-8D or the heating device may have a shape based on the configuration of the selected elongate track guide. The track guide structure 6 and elongate track guide 7 is not shown in FIG. 1. An insulating layer may e.g. be located between the first wall 3 and the heating device 8.

In FIG. 2, another embodiment of the invention is illustrated. FIG. 2 illustrates a warmer arrangement 1 comprising a set of housing parts 2 with a first housing part 2A and a second housing part 2B. Both housing parts 2 have a wall and four edge portions 4 as described above for FIG. 1. The first housing part comprises a first wall, which is not visible, and four edge portions 4a. The second housing part comprises a second housing part wall 3b, and four second housing part edge portion 4b.

The first housing part 2A and a second housing part 2B are rotatably connected via a rotating device 10, such as a hinge.

The first housing part 2A and the second housing part 2B comprises each a locking element 9, which may be adapted for engage with each other when the first housing part 2A and a second housing part 2B are rotated via the rotating device 10 towards each other, whereby the warmer arrangement may be closed.

A track guide structure 6 is arranged within the edge portions of the first housing part 2A and abuts the edge portions 4a along a circumference of the track guide structure 6. The track guide structure 6 thereby is located onto the non-visible first wall. As explained above, a second wall of the first housing part may be located between the track guide structure and the first wall.

The track guide structure 6 comprises an elongate track guide 7 extending from a first end 11a of the elongate track guide and towards a second end 11b of the elongate track guide 7. In an embodiment, the track guide structure 6 comprises a support plate integrated with or supporting the elongate track guide 7. E.g. as described above. The elongate track guide 7 extends in a path having a meandering shape with a plurality of bends. Depending on the tube diameter and the characteristics of the fluid to be heated inside the tube, the elongate track guide 7 may extend in a different way to allow the fluid inside the tube to be heated to a selected temperature. At the first end 11a of the track guide structure 6 the elongate track guide 7 abuts a first opening 5 and the second end 11b of the track guide structure the elongate track guide 7 abuts a second opening 5. In a variation, the track guide structure comprises a support plate 6a as described above. The support plate 6a may form part of or constitute the second wall. Isolating material may be located between the first wall and the second wall.

When the warmer arrangement 1 is turned on, the set of housing parts 2 are closed onto each other as described above and the first housing part 2A and the second housing part 2B are retained in a connected condition by the locking elements 9, which may reduce loss of heat produced by the heating device 8 as explained above. The heating device 8 is arranged between the track guide structure 6 and the wall 3 of the first housing part 2A and is not visible in FIG. 2.

In an embodiment, the first housing part and the second housing part are locked together or is adapted for being locked together to form an enclosing housing. Advantageously, the at least one locking element is adapted for holding the first housing part and the second housing part in a closed and preferably tensioned condition when the warmer arrangement is in operation.

In FIG. 3, the warmer arrangement of FIG. 2 is shown from a top view. The warmer arrangement 1 comprises a display 12 located on an outward facing side 3a of the first wall 3 of the first housing part 2A. The display 12 may preferably be touch-sensitive and may be used to turn the warmer arrangement on and/or off and optionally to set and/or change a target temperature of fluid exiting the warmer arrangement. The display is conveniently in data connection with a controller.

The skilled person will understand that the display 12 in a variation of the embodiment may be located on the second housing part wall 3b of the second housing part 2B.

With reference to FIG. 4, another embodiment of the invention is shown where the warmer arrangement comprises electric powered signal elements 13 that are illuminating. The structure of the warmer arrangement 1 may be as described in FIG. 2. The electric powered signal elements 13 are located on an outward facing side 3a of the first wall 3 of the first housing part 2A. The electric powered signal elements 13 may be used to signal when the warmer arrangement is ready for use, the selected temperature, and warnings. The selected temperature may be illustrated as one or more signal elements illuminating, in FIG. 4 shown as five signal elements, and where the maximum and/or target heating temperature is reached when all five signal elements are illuminating. The signal elements may also be used as touch-sensitive buttons to power the warmer arrangement on/off and for changing the selected temperature. Sometimes a combination of electric powered signal elements 13 and a display is used.

The skilled person will understand that the signal elements 13 in a variation of the embodiment may be located on the second housing part wall of the second housing part 2B.

In FIGS. 5A-5E, side views of five different variations of a locking element 9 of a warmer arrangement are illustrated. In FIG. 5A two housing parts, a first housing part 2A and a second housing part 2B, are connected and retained by the locking elements 9 having a slide locking mechanism. Each of the housing parts 2A, 2B comprises four edge portions 4, where only one of the edge portions 4a of the first housing part 2A and one of the edge portions 4b of the second housing part 2B are visible. The visible edge portions 4a of the first housing part abuts the visible edge portion 4b of the second housing part. The variations illustrated in FIGS. 5A-5E show variations of the locking elements 9 of different embodiments of the invention arrangements.

FIGS. 6A-6D and 7A-7D illustrate variations of track guide structures 6 with different configurations and paths of the elongate track guide 7. The track guide structure 6 shown in FIG. 6A is similar to the track guide structure shown in FIG. 2. The different configurations shown may have different path lengths, shape and width. For example, as the embodiment shown in FIG. 7A which has an elongate track guide width that is larger than the track guide width of the elongate track guide of FIG. 7B. The track guide width may be selected to be adapted to fit a tube with a selected diameter. FIG. 6C shows an example of an elongate track guide 7 with a fold, i.e. where the path of the elongate track guide crosses itself. FIG. 7B shows an example of an elongate track guide with an accordion bended extension.

FIG. 8A shows a warmer arrangement according to an embodiment of the invention comprising a heating device comprising a heating body 14 located within the interior of the first housing part. The heating body 14 is typically located between the track guide structure, not shown, and the at least one first wall 3a of the first housing part. The heating body in FIG. 8A is shown with electrical connections 15 at the edges of the heating body 14, where the heating body is adapted for being or is electrically connected to a power source or optionally to other heating bodies. FIGS. 8B-8D show different variations of the embodiment of FIG. 8A, where the heating device of the warmer arrangements comprise a plurality of heating bodies with different shapes located within the interior of the housing part 3.The selection of number, shape and arrangement of the heating body or heating bodies may be performed in dependence of the elongate track guide configuration, such as length width, shape and number of the elongate track guide(s) and/or for different applications of the warmer arrangement, such as when cooling may be required.

FIG. 9 shows an embodiment of the warmer arrangement with a fixation element 16. The fixation element 16 may be fixated around a limb of user or to another device. Any fixation means may be applied, such as a Velcro strap, a buckle strap, an elastic band, a snap button, hook fastener and similar.

FIG. 10A illustrates the warmer arrangement with a fixation element 16 where the warmer arrangement is fixated by a fixation element 16 around an arm of a user and where a tube, such as an infusion tube is arranged in the channel of the elongate track guide of the warmer arrangement. FIG. 10B shows a variation of FIG. 10A where two tubes are arranged in the warmer arrangement for heating preferably two different fluids flowing in the tubes.

FIGS. 11A-11D show variations of the warmer arrangement 1 shown in FIG. 9, where one or more power sources 17 is/are connected to the not shown heating device of the warmer arrangement 1. In FIG. 11A, an external power source 17 is electrically connected to the heating device and in FIG. 11B, multiple external power sources 17 are electrically connected through a wire W. In FIGS. 11C and 11D, the power source 17a is located within the housing part of the warmer arrangement 1.

FIG. 12 shows a typical IV system with a warmer arrangement 1, where a section, preferably a length section, of the tube 18 of the IV bag system 19 is arranged in the elongate track guide of the warmer arrangement.

FIG. 13 shows a variation of the setup shown in FIG. 12, where an external power source 17 is electrically connected to the heating device of the warmer arrangement 1 and further to a sensor at or correlated to an IV bag 19 for monitoring a characteristic of the fluid in the IV bag, such as temperature, which allow the controller in the warmer arrangement to select and/or target a temperature of the heating body.

The warmer arrangement illustrated in FIG. 14 comprises a first housing part 22a and a second housing part 22b. The first housing part 22a comprises a first wall 23 and a heating device 26. Between the heating device is applied a thermal insulating layer 21c comprising a heat insulating material, preferably as described above. A track guide structure is applied in contact with the heating device 26.

The track guide structure comprises a support plate 27a and an elongate track guide 27b comprising a channel 27c. In the shown embodiment, the support plate 27a and the elongate track guide 27b are integrated with each other. In a variation thereof, the support plate 27a and the elongate track guide 27b may form individual parts.

The surface (i.e. the first surface) of the support plate 27a facing towards the heating device 26 is in the shown embodiment in full physical contact with the heating device 26 to ensure a high transfer of heat.

In a variation thereof, the first surface of the support plate has a surface path in conformation with the track path of the elongate track guide located at the second side of the support plate and wherein a first portion of the first surface of the support plate comprises the surface path and a second portion of the first surface of the support plate is located outside the first portion of the first surface of the support plate.

In an embodiment, the first portion of the first surface of the support plate is in physical contact with the heating device 26 and the second portion of the first surface of the support plate is not in physical contact with the heating device 26, but may for example be isolated from the heating device 26.

In the channel 27c, a section of a tube 28a, such as an infusion tube is located and a fluid 28b is flowing through the tube 28a for being heated in the warmer arrangement.

The second housing part 22b comprises a second housing part wall 25, here forming an outer wall of the warmer arrangement. A track guide lid cover 29 form part of the second housing part 22b and is connected to or integrated with the second housing part wall 25. In a variation there of, the track guide lid cover may be an individual part located between the first housing part 22a and the second housing part 22b.

When first housing part 22a and the second housing part 22b are locked together, the track guide lid cover 29 and the elongate track guide advantageously form an elongate encasing path for encasing the tube 28a for ensuring a desired fast heating of a fluid 28b flowing in the tube 28a.

The first housing part 22a and the second housing part 22b are locked together e.g. by rotating the parts that the track guide lid cover 29 faces towards the channel 27c e.g. via a rotating device as described above. As explained, the tube 28a is located in the channel 27c. The first housing part 22a and the second housing part 22b are locked together, preferably to ensure that the track guide lid cover pressing towards a length of the tube 28a at least when the fluid 28b to be heated in the tube is flowing in the tube 28a.

The first housing part 22a and the second housing part 22b may advantageously be locked together prior to start the flow of the fluid to be heated in the tube 28a.

Thereby any potential risk of damaging the tube by closing the first housing part 22a and the second housing part 22b to each other may be reduced or practically avoided.

As it can be seen in FIG. 14, the tube 28a is slightly deformed by the pressure exerted by the track guide lid cover 29. By pressure the tube 28a in the channel 27c an enlarged contact and a more intimate contact between the tube 28a and the inner surface of the channel may be obtained, which has shown to significantly increase the heat transfer to the liquid 28b flowing the tube 28a.

As shown in FIG. 14, thermal insulating material 21a is located to heat insulate around the track guide lid cover 29 and thermal insulating material 21b is located to heat insulate around the elongate track guide 27b.

The skilled person will understand that in a variation of the embodiment the thermal insulating materials 21a, 21b is formed as a single thermal insulating material attached to one of the first housing part 22a or the second housing part 22b.

The warmer arrangement illustrated in FIG. 15 comprises a first housing part, a second housing part 32b and a track guide lid cover 39 located between the first housing part and the second housing part 32b that are locked together.

The first housing part comprises a first wall 33, a heating device 26 and a track guide structure. The track guide structure comprises a support plate 37a and an elongate track guide 37b comprising a channel 37c. In the shown embodiment, the support plate 37a and the elongate track guide 37b are integrated with each other.

The channel 37c is a semi-closed channel having semi-closed cross-section with an elongate narrow opening extending along the length of the elongate track guide 37b and into the channel 37c.

In the shown embodiment, the elongate narrow opening is closed with the track guide lid cover 39, which is held in tension by the second housing part 32b.

In the channel 37c, a section of a tube 38a, such as an infusion tube is located and a fluid 38b is flowing through the tube 38a for being heated in the warmer arrangement. As seen the outer surface of the tube 38a is fitting tight and intimately to the inner surface of the channel, thereby ensure a highly fast and effective heat transfer to the fluid 38b flowing in the tube 38a.

Prior to closing the first housing part and the second housing part 32b together and prior to arranging the track guide lid cover 39 to close off the elongate narrow opening into the channel 37c, the tube 38a, preferably in empty or at least partially empty condition may be applied into the channel 37c. Due to the semi-closed channel, the feeding of the length section of the tube into the channel becomes relatively simple e.g. as described above. Thereafter, the track guide lid cover 39 is applied to close off the elongate narrow opening into the channel 37c and the first housing part and the second housing part 32b are closed together. After that, the flow of the fluid 38b may be switched on and adjusted as desired. FIG. 16 shows a track guide structure and a track guide lid cover 49.

The track guide structure comprises a support plate 47a and an elongate track guide 47b comprising a channel in which a tube 48 is applied. As it can be seen, the tube 48 protrudes beyond the edges of the channel. When applying the track guide lid cover 49 onto the track guide structure, the track guide lid cover 49 will apply a pressure towards the tube 48, so that the tube will be pressed in tight contact with the inner surface of the channel e.g. as described above.

FIGS. 17a and 17b shows a warmer arrangement comprising a track guide structure, a track guide lid cover 59, and a second housing part 52. The second housing part 52 is closed together with a first housing part where only the track guide structure of the second housing part is shown.

The track guide structure comprises a support plate 57a and an elongate track guide 57b comprising a channel in which a tube 58 is located. The track guide lid cover 59 is located between the second housing part 52 and the track guide structure to provide a pressure towards the tube 58 in the channel to ensure that the tube will be pressed in tight contact with the inner surface of the channel e.g. as described above.

FIG. 18 shows a track guide structure comprising a support plate 67a and an elongate track guide 67b comprising a channel 67c. The elongate track guide 67b is meandering so that several locations of the elongate track guide 67b are seen.

The elongate track guide may have a profile with a radius of 0.2 mm to 4 cm as described above, e.g. where the radius is determined as the radius of a circle C fitting at a bottom portion of the channel as shown in FIG. 18.

FIG. 19 shows a warmer arrangement comprising a first housing part and a second housing part 72B. At the second housing part 72B a locking arrangement is arranged to allow the second housing part 72B to engage with the first housing part and retain locked when the locking arrangement is releasably attached to the first housing part.

The warmer arrangement also comprises a track guide structure having a first surface comprising two protruding elements 71. In a variation thereof, the first surface of the track guide structure may comprise one protruding element or more than two protruding elements 71. The protruding elements are arranged at one or more bends in the channel to provide a more efficiently insertion of the tube in the channel minimizing the risk of the tube falling out under an insertion procedure. Advantageoulsy, the protruding elements are shaped to allow the tube to be at least partly retained in the channel under the insertion procedure.

In a variation thereof, the protruding elements 71 have different selected heights. Typically, a corresponding recess is formed in the second housing part 72B to preferably to ensure that the track guide lid cover pressing towards a length of the tube at least when the fluid to be heated in the tube is flowing in the tube.

FIGS. 20A-20C shows different variations of a cartridge 81. FIG. 20A shows a cartridge 81 comprising two apertures 84a, 84b that allow fluid to enter and discharge from the cartridge. The cartridge comprises a cartridge housing 87 which defines the internal chamber of the cartridge in which the fluid flows. Each of the apertures are connected to a tube comprising a tube connector 82a, 82b. The tube connectors allow the tube and thereby the cartridge to be connected to another tube, preferably an IV tube of an IV system.

In a variation thereof, as shown in FIG. 20B, the two apertures of the cartridge 81 comprises cartridge connectors 83a, 83b, which allow a direct connection between an external tube and the cartridge 81.

In a further variation of the embodiment shown in FIG. 20A, FIG. 20C shows a cartridge 81 comprising two apertures, wherein the aperture allowing discharge of fluid from the cartridge 81 is connected to a tube comprising a tube connector 82a and the aperture that allow fluid to enter the cartridge 81 is connected to a tube directly connected to a drip chamber.

A cartridge of the invention may also be any other combination of the shown embodiments, such as a cartridge comprising a cartridge connector at one aperture and a tube connected directly to a drip chamber at the other aperture.

FIG. 21 shows a warmer arrangement with a first housing part 2A and a second housing part 2B. The warmer arrangement comprises comprising a cartridge 81 which is removably inserted in the first housing part 2A. The cartridge comprises two cartridge connectors 82A and 82B arranged at two apertures and allow a tube to be connected to the cartridge to allow fluid to flow through the cartridge.

The cartridge 81 comprises a cartridge housing which defines an internal chamber of the cartridge 81. The internal chamber comprises a channel 87c which has a meandering shape in which the fluid flows. When a treatment is finished the cartridge can be removed and a new, preferably sterile, cartridge can be inserted in the warmer arrangement for another treatment, preferably an IV treatment.

FIG. 22 shows a variation of the embodiment shown in FIG. 2, where the warmer arrangement comprises an air detector 91 arranged at one end of the channel 97c for detecting air, preferably in the form of air bubbles, in the fluid flowing through the tube in the channel 97c. In a variation thereof, the warmer arrangement may comprise a crystallized matter detector or a combination of an air detector and a crystallized matter detector.

The warmer arrangement also comprises a fluid flow device 92 arranged at the channel 97c. The fluid flow device 92 comprises a force acting element configured for adjustably constricting the tube in the channel to adjust the fluid flow. The force acting element thereby extend into the channel, preferably when receiving a signal from a controller, to adjust the fluid flow in the tube. In a variation thereof, the fluid flow device comprises at least two force acting elements 93 arranged in parallel or opposite each other to provide a regulation of the fluid flow.

1. A warmer arrangement for heating a fluid flowing through a tube, the warmer arrangement comprises
at least one edge portion,
a first housing part forming at least a part of a housing, wherein the first housing part comprises at least one wall comprising a first wall and one or more edge portions of the at least one edge portion attached to and/or integrated with the first wall, wherein the one or more edge portions of the of the first housing part at least partially surround the first wall, wherein the at least one edge portion form an edge structure comprises at least one pair of openings at least partially through the edge structure,
a track guide structure comprising an elongate track guide, and
a heating device comprising at least one heating body located between the track guide structure and the first wall,
wherein the elongate track guide has a length and comprises a channel extending along at least a part of a length of the elongate track guide, and wherein the elongate track guide extends at least partially from a first opening of the least one pair of openings to a second opening of the at least one pair of openings.

2. The warmer arrangement of claim 1, wherein at least one of the one or more edge portions comprises two pairs of openings, wherein each pair of the two pairs of openings comprise a first opening and a second opening arranged opposite to each other.

3. The warmer arrangement of claim 1, wherein the elongate track guide extends from a first end of the track guide structure to an opposite second end of the track guide structure.

4. The warmer arrangement of any claim 1, wherein the heating body is adapted to abut the track guide structure at least partially.

5. The warmer arrangement of claim 1, wherein the heating body is configured for heating the track guide structure or the elongate track guide by at least one of conduction and convection.

6. The warmer arrangement of claim 1, wherein the track guide structure is a heat conductive track guide structure comprising a heat conductive material selected from the list consisting of iron, silver, copper, steel, gold, aluminum, or a combination, and wherein the heating body comprises an inductive coil adapted to provide an electrical field for heating the conductive track guide structure.

7. The warmer arrangement of claim 1, wherein the heating body comprises an infrared emitting element configured to emit infrared radiation.

8. The warmer arrangement of claim 1, wherein the arrangement comprises a monitor sensor and a display for monitoring and displaying least one condition of a fluid tube arranged in the elongate track guide, wherein the condition comprises a temperature of a fluid in the tube and/or at least one dispensing condition.

9. The warmer arrangement of claim 1, wherein the warmer arrangement comprises a fixation element configured to at least partially fixate the arrangement to a limb of a user or an external device.

10. The warmer arrangement of claim 1, wherein the track guide structure comprises a support plate supporting and/or integrated with the elongate track guide, and wherein at least a portion of the second wall of the first housing part form part of or constitutes the supporting plate.

11. The warmer arrangement of claim 10, wherein the support plate has a first surface with a first surface path, and wherein the elongate track guide extend in a track path, and wherein the first surface path is in conformation with the track path, and wherein the first portion of the first surface of the support plate in physical contact with the at least one heating body comprises the first surface path of the support plate.

12. The warmer arrangement of claim 11, wherein a second portion of the first surface outside the first portion of the first surface of the heating body is not in contact with the heating body.

13. The warmer arrangement of claim 10, wherein the support plate has a second side, wherein the elongate track guide is formed by an elongate elevated structure protruding at a second side of the support plate.

14. The warmer arrangement of claim 1, wherein the warmer arrangement comprises a set of housing parts comprising the first housing part and a second housing part and a track guide lid cover, wherein the track guide structure together with the track guide lid cover form an elongate encasing path, suitable for encasing a length of the tube, and wherein the track guide lid cover is adapted to be located between the first housing part and the second housing part or wherein the track guide lid cover forms part of the second housing part.

15. The warmer arrangement of claim 1, wherein the channel has a semi-closed cross-section perpendicular to a center axis of the channel and with an elongate opening into the channel extending along the length of the elongate track guide, wherein the channel has a maximal width in the cross-section, the elongate opening into the channel has a maximal width in the cross-section and wherein the maximal width of the elongate opening into the channel is from 5 % to 40 % of the maximal width of the channel.

16. The warmer arrangement of claim 14, wherein the elongate encasing path has a first diameter in a first plane orthogonal to the plane of the at least one wall and a second diameter in a second plane orthogonal to the first plane, wherein the first diameter is smaller than the second diameter, such as the first diameter is at least 2% smaller than the second diameter.

17. The warmer arrangement of claim 1, wherein the warmer arrangement comprises a set of housing parts comprising the first housing part and a second housing part, and wherein a thermally insulating material forms part of the second housing part or is located between the second housing part and the track guide structure.

18. The warmer arrangement of claim 1, wherein the track guide structure comprises a cartridge comprising at least two apertures aligned with the at least one pair of openings.

19. The warmer arrangement of claim 18, wherein the cartridge comprises a cartridge housing defining an internal chamber comprising the elongate track guide configured to direct fluid through the cartridge housing.

20. The warmer arrangement of claim 1, wherein the first surface of the track guide structure comprises one or more protruding elements protruding from the first surface of the track guide structure, and wherein the one or more protruding elements forms part of a wall of the channel.

21. The warmer arrangement of claim 20, wherein the one or more protruding elements are arranged at the at least one bend and adapted to at least temporarily retain the tube in the channel of the elongate track guide.

22. The warmer arrangement of claim 1, wherein the warmer arrangement comprises at least one fluid flow device arranged in the channel and/or forming part of the channel for adjusting a liquid flow in the tube, wherein the fluid flow device comprises one or more force acting elements, wherein the one or more force acting elements are configured for adjustably constricting the tube to adjust the fluid flow in the tube.

23. The warmer arrangement of claim 1, wherein the warmer arrangement comprises an air detector and/or a crystallized matter detector, wherein the air detector comprises an acoustic sensing element adapted to detect air in the tube.
